(19) United States
(12) Patent Application Publication
(10) Pub. No.: US 2024/0376040 A1
(43) Pub. Date: Nov. 14, 2024

(54) PROCESS FOR THE PREPARATION OF SAFINAMIDE

(71) Applicant: ZACH SYSTEM, Avrillé (FR)

(72) Inventors: Massimiliano FORCATO, Avrillé (FR); Aurélien LEMERCIER, Avrillé (FR); Gérard COQUEREL, Mont-Saint-Aignan Cedex (FR); Yohann CARTIGNY, Mont-Saint-Aignan Cedex (FR)

(73) Assignee: ZACH SYSTEM, Avrillé (FR)

(21) Appl. No.: 18/691,783

(22) PCT Filed: Jun. 8, 2022
(86) PCT No.: PCT/EP2022/065612

§ 371 Date: Mar. 13, 2024

(30) Foreign Application Priority Data
Sep. 14, 2021 (EP) 21306265.6

(51) Int. Cl. C07C 231/12; A61K 9/16; A61K 31/165; C07C 231/24

(52) U.S. Cl. CPC C07C 231/12; A61K 9/1682; A61K 31/165; C07C 231/24

(57) ABSTRACT

The present invention relates to a process for the preparation of spherical agglomerates of safinamide or a pharmaceutically acceptable salt thereof, to the agglomerates thus obtained and to a process for preparing a pharmaceutical composition including said agglomerates.

### FIELD OF THE INVENTION

The present invention relates to a process for the preparation of safinamide or a pharmaceutically acceptable salt thereof by means of a spherical agglomeration process. The invention also relates to the agglomerates thus obtained, and to a process for the preparation of a pharmaceutical composition containing said agglomerates.

### BACKGROUND OF THE INVENTION

Safinamide ((S)-2-[[4-[(3-fluorophenyl)methoxy]phenyl]methylamino]propanamide) is a highly selective and reversible MAO-B inhibitor that causes an increase in the extracellular levels of dopamine in the striatum. Safinamide is associated with state-dependent inhibition of the voltage dependent sodium channels (Na+) and modulation of the stimulated release of glutamate.

Safinamide methanesulfonate is the active ingredient of an EMA-approved drug (Xadago®) that is administered in the form of film-coated oral tablets. Xadago® is indicated for the treatment of adult patients with idiopathic Parkinson's disease as add-on therapy at a stable dose of levodopa (L-dopa) alone or in combination with other drugs for Parkinson's disease in mid-to late-stage fluctuating patients; safinamide acts by a mechanism of action that is both dopaminergic and non-dopaminergic.

Xadago® tablets are characterized by an immediate release profile, and are obtained by dry compaction of the active ingredient mixed with excipients (internal phase), mixing of the compacted material with further excipients (external phase), compression of the final mixture into tablets and coating the latter with coloured polymer film.

Various processes for preparing safinamide and salts thereof are described in EP-A-3 772 510.

Typically, safinamide or a pharmaceutically acceptable salt thereof is isolated as needle-shape crystals. Such a peculiar particles morphology exhibits low flowability properties, which introduces potential causes of inefficiency and poor robustness in the downstream formulation process to produce oral tablets. Moreover, needle-shape crystals are difficult to handle and process on a large (industrial) scale. An additional grinding step is therefore often required before the active ingredient (API) can be formulated into the desired oral form.

In addition, solid state properties of active pharmaceutical ingredients have a decisive impact on dosage form development, stability as well as in vivo performance of the drug. Micrometric properties of drug particles such as shape and size are essential for the formulation of solid dose unit.

It has now been found that spherical agglomerates of safinamide or a pharmaceutically acceptable salt thereof, preferably safinamide methanesulfonate, can be obtained. Such agglomerates exhibit enhanced flowability properties compared to (non-agglomerated) needle-shape crystals.

### SUMMARY OF THE INVENTION

In one aspect, the invention relates to a process for preparing spherical agglomerates of safinamide or a pharmaceutically acceptable salt thereof which comprises:
a) preparing a suspension of safinamide or a pharmaceutically acceptable salt thereof in a hydrocarbon solvent;
b) adding a bridging liquid to the resulting suspension until aggregation is observed;
c) isolating the spherical agglomerates obtained.

In another aspect, the invention relates to spherical agglomerates of safinamide or a pharmaceutically acceptable salt thereof as obtained by said process.

In another aspect, the invention relates to a pharmaceutical composition obtainable by a process comprising:
a) preparing spherical agglomerates of safinamide or a pharmaceutically acceptable salt thereof by the above-mentioned process;
b) adding the resulting spherical agglomerates to one or more pharmaceutically acceptable excipients.

### BRIEF DESCRIPTION OF THE FIGURES

The invention is illustrated by reference to the accompanying drawings described below:

FIG. 1A shows particles of safinamide methanesulfonate before agglomeration.

FIG. 1B shows individualized particles from agglomerates of safinamide methanesulfonate obtained according to the process of the invention.

FIGS. 2A and 2B (cross-section) show SEM pictures of agglomerates of safinamide methanesulfonate obtained according to the process of the invention.

The picture in FIG. 1B was obtained using a KH-7700 Digital Microscope from the company HIROX (lens information provided in the picture legend). The pictures in FIGS. 1A, 2A and 2B were obtained using a JCM-5000 NeoScope™ Scanning Electron Microscope from the company JEOL (magnification ×10 to ×20,000).

FIG. 3 shows the powder X-ray diffractogram of crystalline safinamide methanesulfonate as available under the trade name Xadago® (grey/bottom curve), and as obtained by the spherical agglomeration process of the invention (yellow/top curve).

The relative intensity of the X-ray powder diffraction peaks can vary depending upon sample preparation technique, sample mounting procedure and the particular instrument employed.

The X-ray diffractograms of crystalline safinamide methanesulfonate were measured on a D8 Discover diffractometer (Bruker) equipped with a Cu K alpha radiation source and a Lynx Eye detector (Bruker).

### DETAILED DESCRIPTION OF THE INVENTION

The present invention provides an efficient process to prepare agglomerates of micronic particles of safinamide or of a pharmaceutically acceptable salt thereof by means of a spherical agglomeration process.

It has indeed been found that agglomerates of safinamide or a pharmaceutically acceptable salt thereof can be prepared by a process comprising:
a) preparing a suspension of safinamide or a pharmaceutically acceptable salt thereof in a hydrocarbon solvent;
b) adding a bridging liquid to the resulting suspension until aggregation is observed;
c) isolating the spherical agglomerates obtained.

The process of the invention consists in a spherical agglomeration of safinamide or a pharmaceutically acceptable salt thereof, preferably safinamide methanesulfonate, by using a two solvents system. It involves dispersion of safinamide or a pharmaceutically acceptable salt thereof in a good solvent, namely a hydrocarbon solvent, and agglomeration in the presence of a bridging liquid which acts as an interparticle binder that promotes agglomeration. Said bridging liquid, which should not be miscible with the hydrocarbon solvent, and exhibit greater wettability properties towards safinamide particles, acts like a glue, allowing the particles of safinamide, or a pharmaceutically acceptable salt thereof, suspended in the hydrocarbon solvent to stick together by forming liquid bridges between said particles due to capillary negative pressure and interfacial tension at the solid-liquid interface.

Pharmaceutically acceptable salts of safinamide according to the present invention include addition salts with inorganic acids, for example nitric, hydrochloric, sulphuric, perchloric or phosphoric acid, or with organic acids, for example acetic, propionic, glycolic, lactic, oxalic, malonic, malic, tartaric, citric, benzoic, cinnamic, mandelic, methanesulphonic or salicylic acid.

In some embodiments, the pharmaceutically acceptable salt of safinamide is the methanesulfonate salt.

Safinamide or a pharmaceutically acceptable salt thereof, preferably safinamide methanesulfonate, is first suspended in a hydrocarbon solvent (step a).

In some embodiments, the hydrocarbon solvent is a C₅-C₁₃ linear or branched alkane or a C₅-C₇ cycloalkane. In some embodiments, the hydrocarbon solvent is a C₅-C₁₀ linear or branched alkane. In some embodiments, the C₅-C₁₀ linear or branched alkane includes pentane, hexane, heptane, decane, their isomers and mixtures thereof.

In some embodiments, the C₅-C₁₀ linear or branched alkane includes hexane, heptane, their isomers and mixtures thereof. In some embodiments the C₅-C₁₀ linear or branched alkane includes n-pentane, n-hexane, n-heptane and n-decane. In some embodiments, the C₅-C₁₀ linear or branched alkane includes n-hexane and n-heptane.

In some embodiments, the C₅-C₁₀ linear or branched alkane is n-heptane. In some embodiments, the hydrocarbon solvent is a C₅-C₇ cycloalkane. In some embodiments, the C₅-C₇ cycloalkane includes cyclohexane and methyl cyclohexane.

In some embodiments, the C₅-C₇ cycloalkane is cyclohexane.

In some embodiments, step a) is carried out at a temperature in the range from about 20° C. to about 50° C., such as for example 25° C., 30° C., 35° C. or 40° C.

In some embodiments, the suspension is prepared over a period of time in the range from about 30 min to about 3 h.

In some embodiments, the amount of hydrocarbon solvent is in the range from about 8 to about 30 L/kg, preferably from about 8 to about 15 L/kg.

Once the suspension of safinamide or a pharmaceutically acceptable salt thereof, preferably safinamide methanesulfonate, has been prepared, a bridging liquid is then added to the suspension (step b).

In some embodiments, the bridging liquid is a polar solvent, immiscible with the suspension solvent. In some embodiments, the bridging liquid is a polar solvent, such as a polar aprotic solvent. In some embodiments the polar aprotic solvent includes acetonitrile and propionitrile In some embodiments the polar aprotic solvent is acetonitrile.

In some embodiments, the ratio of the hydrocarbon solvent to the bridging liquid is in the range from about 18:1 v/v to about 8:1 v/v. In some embodiments, the ratio of the hydrocarbon solvent to the bridging liquid is in the range from about 18:1 v/v to about 10:1 v/v.

In some embodments, step b) is carried out at a temperature in the range from about 20° C. to about 50° C., such as for example 25° C., 30° C., 35° C. or 40° C. In some embodiments, the temperature used in step b) is different from that used in step a), (although in the same range). In some embodiments, the temperature used in step b) is identical to that used in step a).

In some embodiments, the bridging liquid is added to the suspension over a period of time in the range from about 30 min to about 2 h.

In some embodiments, the reaction is carried out for a period of time in the range from about 1 h to about 8 days, such as for example in the range from about 1 h to about 3 days, from about 1 h to about 1 day, from about 1 h to about 12 h, or from about 1 h to about 8 h, after the bridging liquid has been added.

In some embodiments steps a) and b) are carried out under anhydrous conditions.

In some embodiments the reaction in step a) and/or in step b) is carried out under stirring. In some embodiments, stitting is performed with a dispersion turbine at a speed in the range of about 250 rpm to about 600 rpm, such as about 350 rpm to about 600 rpm or about 350 rpm to about 500 rpm.

At the end of the reaction, spherical agglomerates of safinamide or a pharmaceutically acceptable salt thereof are isolated (step c).

In some embodiments, the spherical agglomerates of safinamide or a pharmaceutically acceptable salt thereof are isolated by filtration. In some embodiments, the filtered agglomerates are dried, for example up to a temperature of about 50° C., optionally under vacuum.

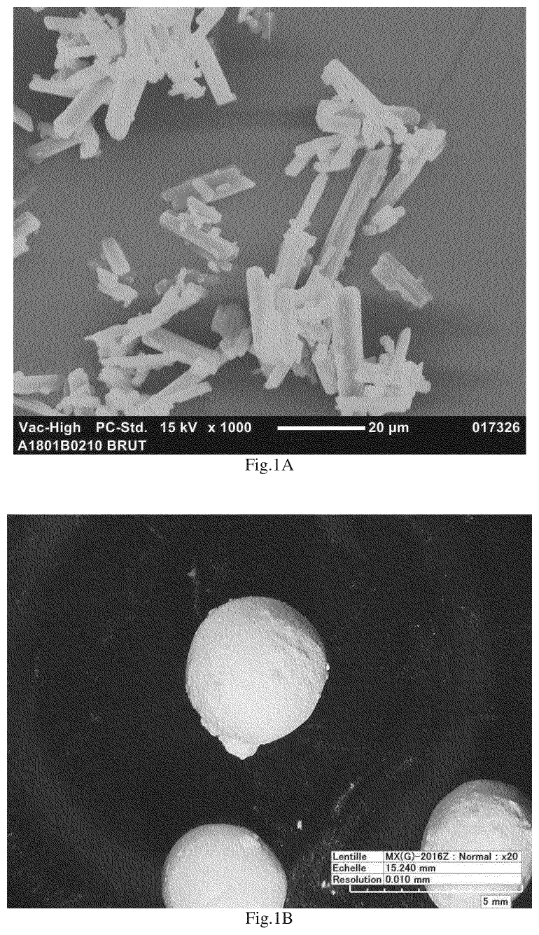

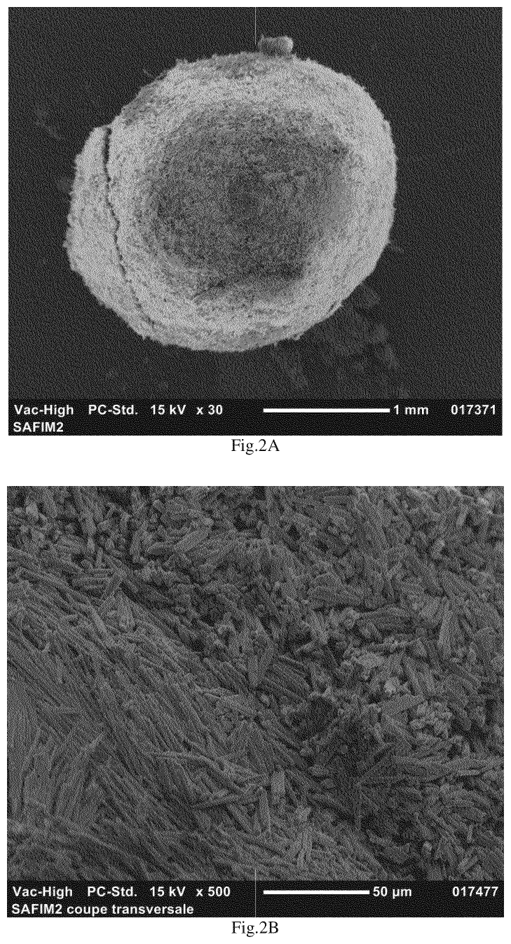

In some embodiments, agglomerates of safinamide or a pharmaceutically acceptable salt thereof, preferably safinamide methanesulfonate, are obtained with a diameter of ≤1 cm. In some embodiments, agglomerates of safinamide methanesulfonate are obtained with a diameter of ≤1 mm. In some embodiments agglomerates of safinamide methanesulfonate are obtained with a diameter in the range from about 100 micrometers to about 500 micrometers, with individual particles of safinamide methanesulfonate having an average particle size of a few microns (see FIG. 1B).

The process described above allows large agglomerates of safinamide or a pharmaceutically acceptable salt thereof to be obtained. The resulting powders are easier to handle due to their granular shape. In addition, the agglomerates easily break down to primary fine particles once they enter physiologic media, thus having limited or no impact on the bioavailability of the drug substance. It is also worth noting that the agglomeration process of the invention does not have an impact on the polymorphic and crystalline form of the active ingredient used as substrate.

Thus an aspect of the invention relates to spherical agglomerates of safinamide or a pharmaceutically acceptable salt thereof, preferably safinamide methanesulfonate, obtained by the process described above. The agglomerates can be used in the preparation of pharmaceutical compositions.

Another aspect of the invention therefore relates to a pharmaceutical composition obtainable by a process comprising:
a) preparing spherical agglomerates of safinamide or a pharmaceutically acceptable salt thereof by the process described above;
b) adding the resulting spherical agglomerates to one or more pharmaceutically acceptable excipient(s).

The choice of excipient(s) will to a large extent depend on factors such as the particular mode of administration, the effect of the excipient on solubility and stability, and the nature of the dosage form.

Pharmaceutical compositions can be prepared by conventional methods, as described e.g. in Remington's Pharmaceutical Sciences, 19^{th} Edition (Mack Publishing Company, 1995), incorporated herein by reference.

In some embodiments, the pharmaceutical composition is an oral dosage form.

The process described above industrial production, and constitutes an efficient and economic alternative for the preparation of safinamide or a pharmaceutically acceptable salt thereof.

The inventors have developed an original process which avoids micronization via conventional physical means i.e. by micronizers or fluid-energy impact equipments and makes it possible to obtain the API, preferably safinamide methanesulfonate, with a size suitable for subsequent formulation into a pharmaceutical dosage form.

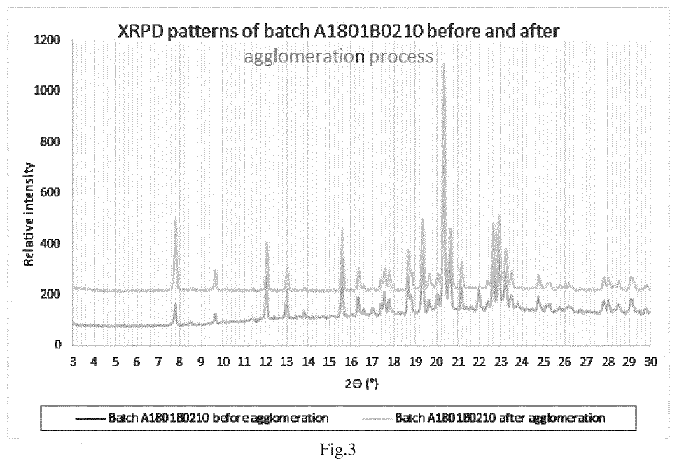

As can be seen from FIG. 3, the agglomerates of safinamide methanesulfonate obtained exhibit a XRD diffraction pattern which is consistent with that of safinamide methanesulfonate in the approved drug Xadago®.

In substance, the process of the invention provides for:
a) easy and efficient isolation of the API;
b) no micronization by micronizers or fluid-energy impact equipments needed;
c) reduced physical stress of particles;
d) reduced confinement concerns;
e) no polymorphic nor crystalline form conversion concerns.

The invention will be better understood in the light of the examples below given by way of illustration.

### Example 1

A solution of 1.00 kg of Safinamide (3.31 mol) in 8.2 L of acetone and 0.6 L of demineralized water was heated to 45±5° C. and rinsed with 0.4 L of acetone. The solution was heated to reflux for 30 minutes, then 0.32 kg of methanesulfonic acid (3.327 mol; 0.99 eq.) was added over 20 minutes. The temperature was maintained at 55±5° C. 0.12 L of demineralised water was added. The temperature was adjusted to 20±2° C. and maintained for 45 minutes or until the fluidisation of the suspension. The suspension was cooled to −7±5° C., kept at that temperature for 2 h, filtered and washed with 2.0 L of acetone at 5±5° C. The resulting solid was dried under vacuum at 40° C. to obtain safinamide methanesulfonate.

Safinamide methanesulfonate was then recrystallized as follows. 1 kg of safinamide methanesulfonate was dissolved in 8.2 L of acetone and 0.8 L of demineralized water. The solution was heated to 45±5° C. for 30 minutes, clarified and cooled to 20±2° C. The reaction medium was stirred until fluidization and cooled to −7±5° C. The resulting suspension was filtered after 2 h and rinsed with 2 L of acetone. The wet residue was then dried under vacuum at 40° C. to give pure safinamide methanesulfonate (99.95%) in 75% yield.

### Example 2

In a 500 mL crystallizer, 30.30 g of the product obtained in Example 1 were added to 300 mL of n-heptane (HPLC grade) at 30° C. The system was then stirred at 425 rpm with a dispersion turbine. After 1 hour of equilibration, 25 mL of acetonitrile (HPLC grade) were added dropwise over 1 hour (volume ratio of n-heptane to acetonitrile=12). The reaction medium was stirred under the same conditions (425 rpm, dispersion turbine) for another 6 h after acetonitrile was added.

The evolution of the system was monitored at different times (2 h, 5 h). After 2 h of stirring, a product was agglomerated. The presence of solid stuck on the wall of the crystallizer was noted.

5 hours after the addition of acetonitrile, the reaction medium was partially deagglomerated and more solid adhered to the wall. 1 h later, the reaction medium was filtered. Rather large agglomerates of safinamide methanesulfonate (0.5 to 1 cm in diameter) were obtained after drying under vacuum at 30° C.

### Example 3

The procedure of example 2 was repeated using 30.00 g of the product obtained in Example 1 and 20 mL of acetonitrile (volume ratio of n-heptane to acetonitrile=15). Agglomerates of safinamide methanesulfonate (500 micrometers in diameter) were obtained after drying under vacuum at 30° C.

### Example 4

In a 500 mL crystallizer, 21.00 g of the product obtained in Example 1 were added to 300 mL of n-heptane (HPLC grade) at 30° C. The system was then stirred at 475 rpm with a dispersion turbine. After 1 hour of equilibration, 22 mL of acetonitrile (HPLC grade) were added dropwise over 1.1 hour (volume ratio of n-heptane to acetonitrile=13.6). The reaction medium was stirred under the same conditions (475 rpm, dispersion turbine) for another 2 h after acetonitrile was added.

After 2 h of stirring, a product was agglomerated. Small agglomerates of safinamide methanesulfonate (700 μm in diameter) and larger agglomerates (1000 μm in diameter) were obtained after drying under reduced pressure at 45° C.

### Example 5

In a 500 mL crystallizer, 21.00 g of the product obtained in Example 1 were added to 300 mL of n-heptane (HPLC grade) at 30° C. The system was then stirred at 400 rpm with a dispersion turbine. After 1 hour of equilibration, 25 mL of propionitrile (HPLC grade) were added dropwise over 1.5 hour (volume ratio of n-heptane to propionitrile=12). The reaction medium was stirred under the same conditions (400 rpm, dispersion turbine) for another 2 h after acetonitrile was added.

After 2 h of stirring, a product was agglomerated. Agglomerates of safinamide methanesulfonate (1000 μm in diameter) were obtained after drying under reduced pressure at 45° C.

### Example 6

In a 500 mL crystallizer, 21.00 g of the product obtained in Example 1 were added to 300 mL of methyl cyclohexane (HPLC grade) at 30° C. The system was then stirred at 400 rpm with a dispersion turbine. After 1 hour of equilibration, 25 mL of acetonitrile (HPLC grade) were added dropwise over 1.5 hour (volume ratio of methyl cyclohexane to acetonitrile=12). The reaction medium was stirred under the same conditions (400 rpm, dispersion turbine) for another 2 h after acetonitrile was added.

After 2 h of stirring, a product was agglomerated. Agglomerates of safinamide methanesulfonate (1000 μm in diameter) were obtained after drying under reduced pressure at 45° C.

### Comparative Example 1

The procedure of example 2 was repeated using 30.00 g of the product obtained in Example 1 and 15 mL of acetonitrile (volume ratio of n-heptane to acetonitrile=20). No agglomerates of safinamide methanesulfonate were obtained.

### Comparative Example 2

The procedure of example 2 was repeated using 30.00 g of the product obtained in Example 1 and 60 mL of acetonitrile (volume ratio of n-heptane to acetonitrile=5). No agglomerates of safinamide methanesulfonate were obtained.

1: A process for preparing spherical agglomerates of safinamide or a pharmaceutically acceptable salt thereof, comprising:
a) preparing a suspension of safinamide or a pharmaceutically acceptable salt thereof in a hydrocarbon solvent;
b) adding a bridging liquid to the resulting suspension until aggregation is observed;
c) isolating the spherical agglomerates obtained.

2: The process of claim 1, wherein the hydrocarbon solvent is a C₅-C₁₃ linear or branched alkane or a C₅-C₇ cycloalkane.

3: The process of claim 2, wherein the solvent is a C₅-C₁₃ alkane which is n-hexane or n-heptane.

4: The process of claim 2, wherein the solvent is a C₅-C₇ cycloalkane which is cyclohexane or methyl cyclohexane.

5: The process of claim 1, wherein preparing a suspension a) is carried out at a temperature in the range from about 20 to about 50° C.

6: The process of claim 1, wherein the bridging liquid is a polar aprotic solvent.

7: The process of claim 6, wherein the polar aprotic solvent is acetonitrile or propionitrile.

8: The process of claim 1, wherein the temperature of adding a bridging liquid b) is identical to the temperature of preparing a suspension a).

9: The process of claim 1, wherein the temperature of adding a bridging liquid b) is within the same range, but different, from the temperature of preparing a suspension a).

10: The process of claim 1, wherein in b) the bridging liquid is added to the suspension over a period of time in the range from about 30 min to about 2 h.

11: The process of claim 1, wherein a) and b) are carried out under anhydrous conditions.

12: The process of claim 1, wherein isolating the spherical agglomerates obtained c) is carried out by filtration to obtain a solid.

13: The process of claim 12, wherein the resulting solid is dried.

14: The process of claim 1, wherein a ratio of the hydrocarbon solvent to the bridging liquid is in the range from about 18:1 v/v to about 8:1 v/v.

15: The process according to claim 1, wherein a pharmaceutically acceptable salt of safinamide is prepared, and the pharmaceutically acceptable salt is safinamide methanesulfonate.

16: Spherical agglomerates of safinamide or a pharmaceutically acceptable salt thereof obtained by the process of claim 1.

17: A pharmaceutical composition obtained by a process comprising:
a) preparing spherical agglomerates of safinamide or a pharmaceutically acceptable salt thereof by the process of claim 1;
b) adding the spherical agglomerates to one or more pharmaceutically acceptable excipients.

18: The pharmaceutical composition according to claim 17, wherein said composition is an oral dosage form.
